(19) United States
(12) Patent Application Publication
(10) Pub. No.: US 2022/0112515 A1
(43) Pub. Date: Apr. 14, 2022

(54) Method for Producing Virus-Infected Cell Line and Animal Model

(71) Applicants: INDUSTRY-ACADEMIC COOPERATION FOUNDATION, YONSEI UNIVERSITY, Seoul (KR); UNIVERSITY INDUSTRY FOUNDATION, YONSEI UNIVERSITY WONJU CAMPUS, Gangwon-do (KR)

(72) Inventors: Hong Jai Lee, Seoul (KR); Soo Ki Kim, Seoul (KR); Jae Seung Moon, Seoul (KR); Bo Young Shin, Daegu (KR); Jin Su Shin, Seoul (KR); Sang Kyou Lee, Seoul (KR)

(73) Assignees: INDUSTRY-ACADEMIC COOPERATION FOUNDATION, YONSEI UNIVERSITY, Seoul (KR); UNIVERSITY INDUSTRY FOUNDATION, YONSEI UNIVERSITY WONJU CAMPUS, Gangwon-do (KR)

(21) Appl. No.: 17/288,253

(22) PCT Filed: Oct. 25, 2019
(86) PCT No.: PCT/KR2019/014153

§ 371 Date: Apr. 23, 2021

(30) Foreign Application Priority Data
Oct. 25, 2018 (KR) 10-2018-0128199
Oct. 25, 2018 (KR) 10-2018-0128201

(51) Int. Cl. C12N 15/85; C12N 7/00; A01K 67/027

(52) U.S. Cl. CPC C12N 15/8509; C12N 7/00; A01K 67/027; A01K 2227/105; C12N 2730/10111; C12N 2015/8581; A01K 2267/0337; C12N 2770/24211

(57) ABSTRACT

Disclosed herein are methods for producing virus-infected cell lines or animal models, wherein an enveloped virus including a lipid bilayer is mixed with a bile acid or a bile acid derivative, which allows the lipid bilayer to be replaced with a lipid bilayer derived from a target animal. Also disclosed herein are the virus-infected cell lines or animal models so produced and methods of screening a therapeutic candidate for a viral disease using the same.

### CROSS-REFERENCE TO RELATED APPLICATIONS

This application is U.S. national phase application under 35 U.S.C. § 371 of International Application No. PCT/KR2019/014153, filed on Oct. 25, 2019, which claims the benefit of priority to Korean Patent Application No. KR10-2018-0128199, filed on Oct. 25, 2018, and Korean Patent Application No. KR10-2018-0128201, filed on Oct. 25, 2018, the contents of each of which are incorporated herein by reference in their entirety.

### FIELD

The present disclosure generally relates to in vitro and in vivo models for viral infection studies. More specifically, the present disclosure relates to a method of producing virus-infected cell lines or virus-infected animal models using an enveloped virus whose lipid bilayer has been replaced with a lipid bilayer of a target animal.

### BACKGROUND

In general, virion, which is a viral particle that has the ability to infect host cells, includes a core genome containing genetic material such as DNA or RNA, and a capsid, which is a protein shell that functions to protect such genetic material, and further includes an envelope in some cases. Types of viruses including an envelope include DNA viruses such as herpesvirus; RNA viruses such as flavivirus and retrovirus; and the like.

Viruses that cause hepatitis (hepatitis viruses) may be classified into enveloped viruses (types B, C, and D) and non-enveloped viruses (types A and E). Hepatitis B virus (hereinafter referred to as ‘HBV’), which is estimated to have an infection rate of about 350 million people worldwide, is a typical virus including an envelope and is a major cause of chronic liver disease. The HBV is a virus having a DNA genome that belongs to the hepadnavirus family and causes acute and chronic hepatitis. In particular, the prevalence of HBV, which was such that a percentage of chronically infected patients reached about 5% to 8% in Korea and China, has been decreased to some extent due to the development of vaccines. However, the truth is that a large number of chronic hepatitis patients still exist due to continuing outbreak of the mother-to-child vertical HBV infection. Chronic HBV infection causes liver cancer as well as hepatitis and cirrhosis. The WHO's research has demonstrated that about 80% of liver cancers is caused by chronic hepatitis B, and the incidence of liver cancer is about 300 times higher in HBV-infected people than non-HBV-infected people.

The HBV is classified into 8 genotypes whose genetic base sequences differ from one another by about 8% or higher, or 4 types of serotypes (adw, adr, ayw, and ayr, or the like) based on the two epitopes (d/y, w/r) in HBV surface antigen (HBsAg). Since the liver, which is a host of the hepatitis virus, induces immune tolerance, the virus can survive without rejection even in a case of histocompatibility complex-mismatched liver allografts.

On the other hand, for the hepatitis C virus (hereinafter referred to as “HCV”), about 170 million people worldwide are infected with this virus. A high infection rate thereof is seen mainly in developed countries; and the HCV infection is highly associated with tattoos, administration of drugs such as narcotic drugs, and AIDS infection. Most HCV infections take a chronic course, and cause cirrhosis and liver cancer as complications thereof. HCV, which is an RNA genome virus, is a hepacivirus belonging to the Flaviviridae family and is divided into six genotypes (types 1 to 6), each genotype being divided into various subtypes. These subtypes of HCV are further differentiated in patients infected with HCV, and have quasispecies that exhibits genetic diversity.

In a case of chronic HBV infection, treatment is performed by oral administration of a nucleoside analog to inhibit viral proliferation, or by injection of interferon into an infected patient. The nucleoside analog has a better therapeutic effect than interferon, and has problems in that chronic use of the nucleoside analog causes HBV to develop resistance thereto and may also cause HBV to develop cross-resistance to other drugs. In addition, in a case of HCV infection, interferon monotherapy was attempted in the 1990's, and a treatment success rate thereof was only 10%. In the 2000s, combination therapy of peginterferon alpha and ribavirin resulted in an approximately 50% improvement in treatment success rate; however, this therapy caused severe drug adverse effects, and thus there was a difficulty in achieving practical treatment. Recently, direct antiviral agents (DAAs) have been developed, and oral administration of such drugs for 3 to 6 months made it possible to obtain a treatment success rate of about 80% to 90% with little adverse effects. However, since the DAA drugs are very expensive, there are problems that such drugs become a barrier to treatment for many patients and cause a huge loss of social costs. In addition, the HBV and HCV therapeutic agents developed so far are problematic in that such therapeutic agents do not ultimately kill the viruses but merely inhibit proliferation thereof.

The reason why no drugs capable of ultimately killing HBV and HCV have been developed to date is that there is no method of actively culturing viruses with an in vitro cell culture system. In addition, there is no method available which enables production of an animal model by infection of small animals having a small size, such as mice, which can be traditionally used in infection experiments.

Due to such circumstances, for viruses, the life cycle of viral infection has not been accurately identified. In this regard, for HBV, the mechanism that can destroy cccDNA, which is a template for intranuclear viral genome replication, has not been elucidated; and for HCV, the mechanism for quasispecies that exhibits genetic diversity in infected patients has not been elucidated. For this reason, a perfect and ideal antiviral drug has not yet been developed.

The viruses, such as HBV and HCV, which include a lipid bilayer envelope, exhibit species specificity (or narrow host range) and organ tropism. This made it difficult to achieve viral infection and culture in vitro and in small animals. Thus, so far, there has been no progress in development of therapeutic agents for the viruses.

In the past few years, in the development of animal models for HBV and HCV, significant progress has been made such as construction of chimeric animal models capable of infection with the viruses. Nevertheless, lack of animal models presents a problem in that there is no clinically appropriate means for developing therapeutic agents suitable for viral hepatitis diseases. In particular, since HBV and HCV use only humans and chimpanzees as hosts, there is a problem that the viruses have such a limited host range, which presents a particular difficulty in producing in vivo models as well as in vitro models.

Therefore, there is a need for in vitro (e.g., cell lines) and small animal models as a tool to elucidate the life cycle and mechanism of pathogenesis of hepatitis viruses. The present disclosure fulfills this need.

### SUMMARY

The present disclosure provides, in part, effective in vitro and in vivo models for viral infection studies. More specifically, the present disclosure provides virus-infected cell lines and virus-infected animal models for studies of life cycle and mechanism of pathogenesis of viruses such as hepatitis viruses as well as methods of producing the same. The present disclosure also provides methods of screening therapeutic candidates for viral infections or viral diseases.

Accordingly, one aspect of the present disclosure provides a method for producing a virus-infected cell line. Such method comprises mixing an enveloped virus including a lipid bilayer with a bile acid or a bile acid derivative, and adding the virus to a culture medium of a cell line to produce a virus-infected cell line.

In one embodiment, the enveloped virus is a hepatitis virus. By way of non-limiting example, the hepatitis virus may be hepatitis B virus (HBV) or hepatitis C virus (HCV).

In one embodiment, the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid, or a combination thereof.

In one embodiment, the cell line is selected from the group consisting of a liver cell line, a lung cell line, a kidney cell line, a muscle cell line, a breast cell line, an immune T cell line, and a reproductive cell line.

In one embodiment, the cell line is derived from human or mouse. By way of non-limiting example, the cell line may be human liver cancer cell line HepG2, mouse liver cancer cell line Hepa1c1c7, human lung cancer cell lines SNU-1327 and A549, mouse lung cancer cell line LA-4, human kidney cancer cell line A498, mouse kidney cancer cell line RAG, human breast cancer cell line MDA-MB-231, mouse breast cancer cell line MTV/TM-011, human immune T cell line Jurkat, mouse immune T cell line EL4, human cervical cancer cell lineHeLa, or mouse testicular cancer cell line LC540.

Another aspect of the present disclosure provides a virus-infected cell line produced by the methods described above and herein.

Still another aspect of the present disclosure provides a method for producing a virus-infected animal model. Such method comprises replacing a lipid bilayer of an enveloped virus with a lipid bilayer derived from a target animal, and injecting the virus for which the lipid bilayer replacement has been achieved into the target animal to produce a virus-infected animal model.

In one embodiment, a lipid bilayer of the enveloped virus is replaced with a lipid bilayer derived from the target animal by mixing the enveloped virus with a bile acid or a bile acid derivative.

In one embodiment, the target animal is a vertebrate animal selected from the group consisting of a mouse, rat, rabbit, bird, cat, dog, pig, sheep, goat, deer, horse, and cattle. By way of non-limiting example, the target animal is a mouse.

In one embodiment, the enveloped virus is a hepatitis virus. By way of non-limiting example, the hepatitis virus may be hepatitis B virus (HBV) or hepatitis C virus (HCV).

In one embodiment, the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid, or a combination thereof.

Yet another aspect of the present disclosure provides a virus-infected animal model, produced by the method described above and herein.

Still yet another aspect of the present disclosure provides a method for screening a therapeutic candidate for a viral disease by adding a therapeutic candidate for a viral disease to the virus-infected cell line described above and herein. In this method, a decreased or reduced presence or absence of the viral genome or surface antigen associated with the viral disease in the virus-infected cell line after the addition of the therapeutic candidate suggests that the therapeutic candidate is an effective agent for the viral disease.

Still yet another aspect of the present disclosure provides a method for screening a therapeutic candidate for a viral disease by administering a therapeutic candidate for a viral disease to the virus-infected animal model described above and herein. In this method, a decreased or reduced presence or absence of the viral genome or surface antigen associated with the viral disease in the virus-infected animal model post administration of the therapeutic candidate suggests that the therapeutic candidate is an effective agent for the viral disease.

For the methods of screening a therapeutic candidate for a viral disease disclosed above and herein, by way of non-limiting example, the viral disease may be viral hepatitis.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates identification of HBcAg of HBV in human liver cancer cell line HepG2 through fluorescence-activated cell sorting.

FIG. 2 illustrates identification of HBcAg of HBV in mouse liver cancer cell line Hepa1c1c7 through fluorescence-activated cell sorting.

FIG. 3 illustrates identification of HBcAg of HBV in human kidney cancer cell line A498 through fluorescence-activated cell sorting.

FIG. 4 illustrates identification of HBcAg of HBV in mouse kidney cancer cell line RAG through fluorescence-activated cell sorting.

FIG. 5 illustrates identification of HBcAg of HBV in human breast cancer cell line MDA-MB-231 through fluorescence-activated cell sorting.

FIG. 6 illustrates identification of HBcAg of HBV in mouse breast cancer cell line MTV/TM-011 through fluorescence-activated cell sorting.

FIG. 7 illustrates identification of HBcAg of HBV in human immune T cell line Jurkat through fluorescence-activated cell sorting.

FIG. 8 illustrates identification of HBcAg of HBV in human cervical cancer cell lineHeLa through fluorescence-activated cell sorting.

FIGS. 9-16 illustrate results obtained by performing H&E staining of organs of a virus-infected animal model.

FIGS. 17-22 illustrate results obtained from immunostaining of liver tissues excised from a virus-infected animal model.

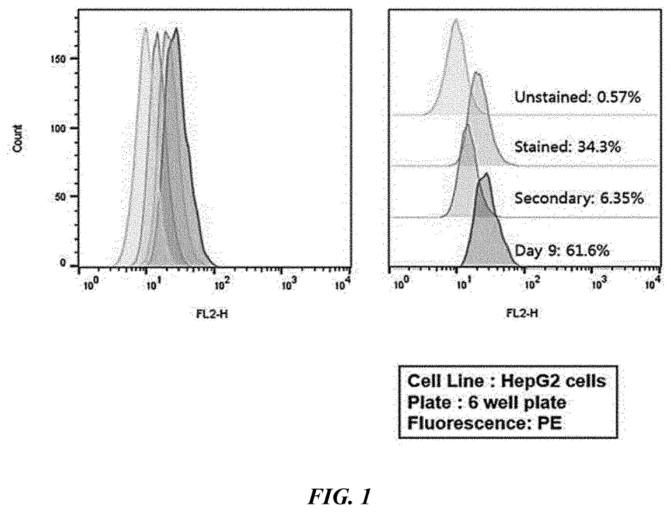

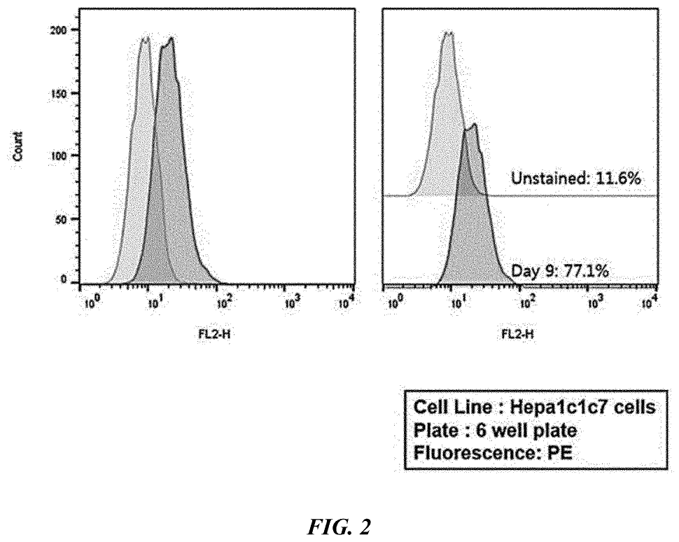

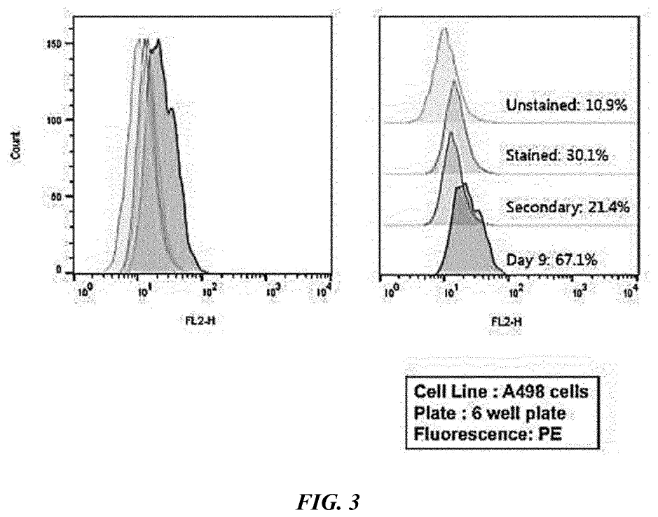

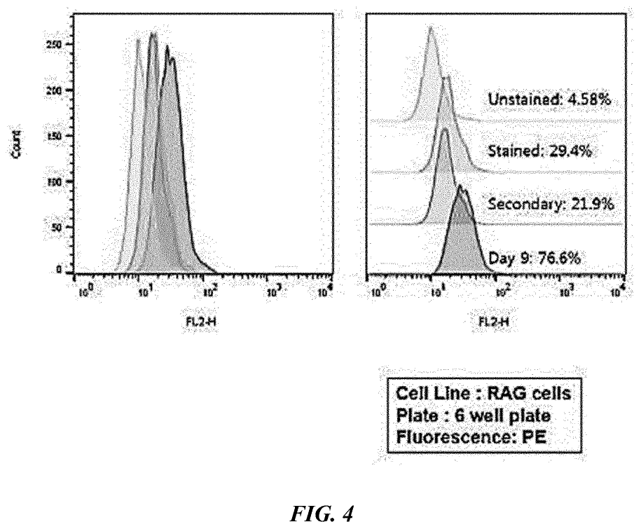

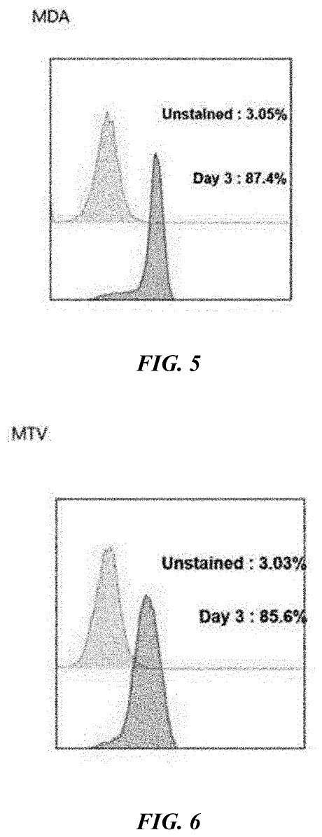

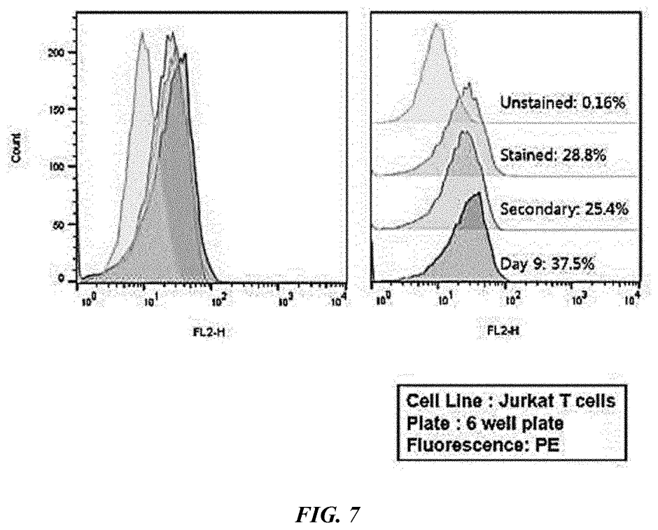

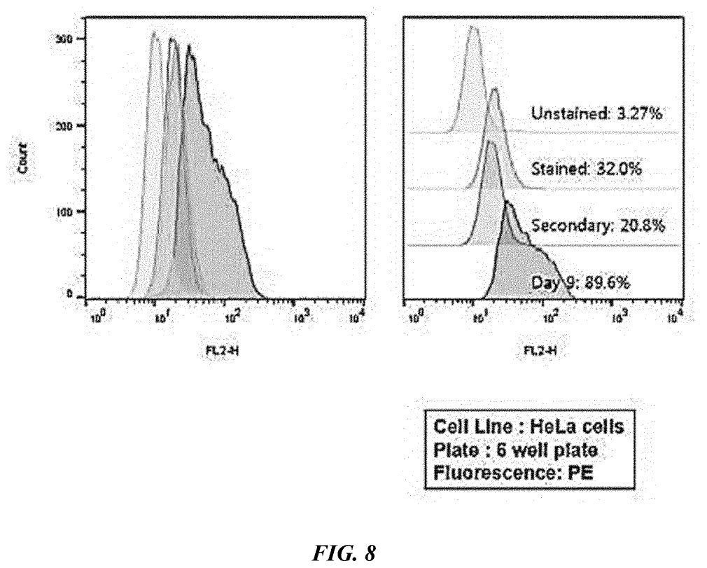

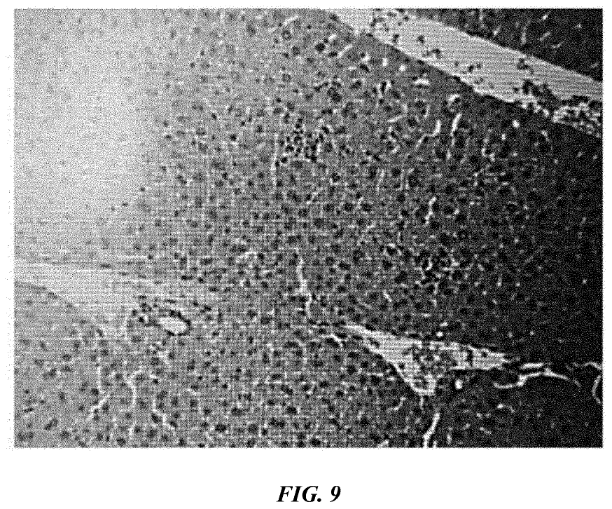

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Section headings as used in this section and the entire disclosure herein are merely for organizational purposes and are not intended to be limiting.

An object of the present disclosure is to provide a method of producing a virus-infected cell line or a virus-infected animal model as well a virus-infected cell line or a virus-infected animal model so produced. Another object of the present disclosure is to provide a method of screening a therapeutic candidate for a viral disease using a virus-infected cell line or a virus-infected animal model. However, the technical problem to be solved by the present disclosure is not limited to the above-mentioned problems. Other problems which are not explicitly mentioned will be clearly understood by those skilled in the art from the following description.

The solution provided by the present disclosure is provision of a virus-infected cell line or a virus-infected animal model as well as methods of producing the same. First of all, the present disclosure has uncovered that in a case where a culture medium of a cell line is treated with a mixture of a bile acid or a bile acid derivative and an enveloped virus, the bile acid mechanically expands the lipid bilayer of the cell line, thereby causing destruction of the cell membrane, so that the viral infection occurs regardless of the virus's species specificity and tissue specificity. Furthermore, the present disclosure has uncovered that in a case where an enveloped virus including a lipid bilayer that has been replaced with a lipid bilayer derived from a target animal is injected into the target animal, it is possible to produce an animal model that can be used clinically for viral infection studies or therapeutic candidate screening.

Accordingly, one aspect of the present disclosure provides a method for producing a virus-infected cell line. Such method comprises mixing an enveloped virus including a lipid bilayer with a bile acid or a bile acid derivative, and adding the virus to a culture medium of a cell line to produce a virus-infected cell line.

As used herein, the term “enveloped virus including a lipid bilayer” refers to an enveloped virus that includes a core genome containing genetic material such as DNA or RNA, and a capsid, which is a protein shell that functions to protect such genetic material; and an envelope containing a lipid bilayer.

The enveloped virus may include any virus as long as the virus further includes an envelope containing a lipid bilayer in addition to a capsid constituting the virus itself. By way of non-limiting example, the enveloped virus may be hepadnavirus, poxvirus, herpesvirus, and the like, which have DNA as genetic material; or coronavirus, filovirus, rhabdovirus, arenavirus, flavivirus, retrovirus, and the like, which have RNA as genetic material. By way of non-limiting example, the virus may be a hepatitis virus such as hepatitis B virus (HBV) and hepatitis C virus (HCV).

As used herein, “a virus-infected cell line” means a cell line that is infected with the enveloped virus including a lipid bilayer. The virus-infected cell line is capable of maintaining its characteristics even in a case of being sub-cultured for a certain period of time in vitro. By way of non-limiting example, the cell line may be immortalized so that it can be continuously sub-cultured in vitro.

The method for producing a cell line infected with an enveloped virus including a lipid bilayer disclosed above and herein comprises the step of adding the virus mixed with the bile acid or bile acid derivative in vitro to a culture medium of a cultured cell line. Conventionally, it was difficult to induce infection of a cultured immortalized cell line with an enveloped virus including a lipid bilayer in vitro; however, in a case where a bile acid or a bile acid derivative is mixed with the virus and the mixture is used to treat a culture medium of the immortalized cell line, the lipid bilayer of the cell line is expanded so that the cell membrane is mechanically destroyed, and this makes it possible to effectively induce infection of the immortalized cell line with the enveloped virus including a lipid bilayer even in vitro.

Due to such an effect, it is possible to produce a virus-infected cell line model using cell lines derived from various species and tissues, without limitation resulting from the viral species specificity and the viral specificity of having tropism to a specific tissue.

For humans, the bile acid is a primary bile acid synthesized from cholesterol in the liver, which is divided into cholic acid and chenodeoxycholic acid; and a second bile acid is produced from the primary bile acid through metabolism by intestinal bacteria in the large intestine, in which cholic acid is metabolized to deoxycholic acid or chenodeoxycholic acid is metabolized to lithocholic acid. In some embodiments, the bile acid may be conjugated with glycine or taurine in the liver, and thus converted into taurocholic acid and glycocholic acid (derivatives of cholic acid), taurochenodeoxycholic acid and glycochenodeoxycholic acid (derivatives of chenodeoxycholic acid), glycodeoxycholic acid and taurodeoxycholic acid (derivatives of deoxycholic acid), or glycolithocholic acid and taurolithocholic acid (derivatives of lithocholic acid). In some embodiments, for rodents, muricholic acid is present as a secondary bile acid.

In one embodiment, the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid. By way of non-limiting example, the bile acid or bile acid derivative may be taurochenodeoxycholic acid or taurocholic acid. By way of non-limiting example, taurochenodeoxycholic acid may be used for HBV, and taurocholic acid or taurochenodioxycholic acid may be used for HCV.

When mixed with the virus, the bile acid or bile acid derivative may be used at a concentration of 10 to 100 μmol/L, preferably 50 to 100 μmol/L, and more preferably 80 to 100 μmol/L. In a case where the bile acid is used at a concentration of lower than 10 μmol/L, the viral envelope may not be sufficiently removed; and in a case where the bile acid is used at a concentration of higher than 100 μmol/L, the viral genome stability and the like may be impaired.

The cell line may be derived from normal or cancerous tissues and sub-cultured in vitro. The cell line may be immortalized according to a conventional method. In one embodiment, the cell line is selected from the group consisting of a liver cell line, a lung cell line, a kidney cell line, a muscle cell line, a breast cell line, an immune T cell line, and a reproductive cell line. In one embodiment, the cell line is derived from human or mouse.

By way of non-limiting example, the human liver-derived cell line may be HepG2 cell line or Huh-7 cell line, and the mouse liver-derived cell line may be Hepa1c1c7 cell line or H4-II-E cell line.

By way of non-limiting example, the human-derived immortalized cell line may be SNU-1327 cell line or A549 cell line (lung cancer cell lines), A-498 cell line (a kidney cancer cell line), MDA-MB-231 cell line (a breast cancer cell line), Jurkat cell line (an immune T cell line), or HeLa cell line (a cervical cancer cell line).

By way of non-limiting example, the mouse-derived immortalized cell line may be LA-4 cell line (a lung cancer cell line), RAG cell line (a kidney cancer cell line), MTV/TM-011 cell line (a breast cancer cell line), EL4 cell line (an immune T cell line), or LC540 cell line (a testicular cancer cell line).

In the virus-infected cell line production methods disclosed above and herein, after mixing the enveloped virus including a lipid bilayer with a bile acid or a bile acid derivative, the enveloped virus including a target animal-derived lipid bilayer may be isolated from the culture medium by a conventional method including, but not limited to, centrifugation and chromatography.

In these methods, the bile acid or bile acid derivative is mixed with an enveloped virus including a lipid bilayer, and the mixture is added to a culture medium of a cell line derived from a target animal model. As a result, the lipid bilayer of the envelope virus is replaced with a lipid bilayer derived from the target animal model, so that the virus is capable of infecting an animal other than humans without species specificity. By way of non-limiting example, a human-derived lipid bilayer of the enveloped virus is replaced with a target animal-derived lipid bilayer, which can facilitate membrane fusion, thereby inducing a cross-species infection.

Another aspect of the present disclosure provides a virus-infected cell line produced by the methods described above and herein. In some embodiments, the virus-infected cell line may be produced by mixing an enveloped virus including a lipid bilayer with a bile acid or a bile acid derivative, and adding the mixture of the virus and the bile acid or bile acid derivative to a culture medium of an immortalized cell line.

Any of the features described above and herein with regard to virus-infected cell line production methods are applicable to the virus-infected cell lines so produced, which include, but are not limited to, the enveloped virus including a lipid bilayer, the bile acid or bile acid derivative, the immortalized cell line.

Still another aspect of the present disclosure provides a method for producing a virus-infected animal model. Such method comprises replacing a lipid bilayer of an enveloped virus with a lipid bilayer derived from a target animal, and injecting the virus for which the lipid bilayer replacement has been achieved into the target animal to produce a virus-infected animal model.

In one embodiment, a lipid bilayer of the enveloped virus is replaced with a lipid bilayer derived from the target animal by mixing the enveloped virus with a bile acid or a bile acid derivative.

The enveloped virus may include any virus as long as the virus further includes an envelope containing a lipid bilayer in addition to a capsid constituting the virus itself. By way of non-limiting example, the enveloped virus may be hepadnavirus, poxvirus, herpesvirus, and the like, which have DNA as genetic material; or coronavirus, filovirus, rhabdovirus, arenavirus, flavivirus, retrovirus, and the like, which have RNA as genetic material. By way of non-limiting example, the virus may be a hepatitis virus such as hepatitis B virus (HBV) and hepatitis C virus (HCV).

A target animal may be a vertebrate animal selected from the group consisting of a mouse, rat, rabbit, bird, cat, dog, pig, sheep, goat, deer, horse, and cattle. By way of non-limiting example, the target animal is a mouse.

For humans, the bile acid is a primary bile acid synthesized from cholesterol in the liver, which is divided into cholic acid and chenodeoxycholic acid; and a second bile acid is produced from the primary bile acid through metabolism by intestinal bacteria in the large intestine, in which cholic acid is metabolized to deoxycholic acid or chenodeoxycholic acid is metabolized to lithocholic acid. In some embodiments, the bile acid may be conjugated with glycine or taurine in the liver, and thus converted into taurocholic acid and glycocholic acid (derivatives of cholic acid), taurochenodeoxycholic acid and glycochenodeoxycholic acid (derivatives of chenodeoxycholic acid), glycodeoxycholic acid and taurodeoxycholic acid (derivatives of deoxycholic acid), or glycolithocholic acid and taurolithocholic acid (derivatives of lithocholic acid). In some embodiments, for rodents, muricholic acid is present as a secondary bile acid.

In one embodiment, the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid. By way of non-limiting example, the bile acid or bile acid derivative may be taurochenodeoxycholic acid or taurocholic acid. By way of non-limiting example, taurochenodeoxycholic acid may be used for HBV, and taurocholic acid or taurochenodioxycholic acid may be used for HCV.

When mixed with the virus, the bile acid or bile acid derivative may be used at a concentration of 10 to 100 μmol/L, preferably 50 to 100 μmol/L, and more preferably 80 to 100 μmol/L. In a case where the bile acid is used at a concentration of lower than 10 μmol/L, the viral envelope may not be sufficiently removed; and in a case where the bile acid is used at a concentration of higher than 100 μmol/L, the viral genome stability and the like may be impaired.

In the virus-infected animal model production methods disclosed above and herein, after mixing the enveloped virus including a lipid bilayer with a bile acid or a bile acid derivative, the enveloped virus including a target animal-derived lipid bilayer may be isolated from the culture medium by a conventional method including, but not limited to, centrifugation and chromatography. In one embodiment, the isolated virus may be injected into a vein of the target animal. By way of non-limiting example, the vain may be a tail vein.

In these methods, the bile acid or bile acid derivative is mixed with an enveloped virus including a lipid bilayer, and the mixture is added to a culture medium of a cell line derived from a target animal model. As a result, the lipid bilayer of the envelope virus is replaced with a lipid bilayer derived from the target animal model, so that the virus is capable of infecting an animal other than humans without species specificity. By way of non-limiting example, a human-derived lipid bilayer of the enveloped virus is replaced with a target animal-derived lipid bilayer, which can facilitate membrane fusion, thereby inducing a cross-species infection.

Yet another aspect of the present disclosure provides a virus-infected animal model, produced by the method described above and herein. In one embodiment, the virus-infected animal model may be produced by replacing a lipid bilayer of an enveloped virus with a lipid bilayer derived from a target animal, and injecting the virus for which the lipid bilayer replacement has been achieved into the target animal. In one embodiment, a lipid bilayer of the enveloped virus is replaced with a lipid bilayer derived from the target animal by mixing the enveloped virus with a bile acid or a bile acid derivative.

Any of the features described above and herein with regard to virus-infected animal model production methods are applicable to the virus-infected animal model so produced, which include, but are not limited to, the enveloped virus including a lipid bilayer and the bile acid or bile acid derivative.

Still yet another aspect of the present disclosure provides a method for screening a therapeutic candidate for a viral disease by adding a therapeutic candidate for a viral disease to the virus-infected cell line described above and herein. In this method, a decreased or reduced presence or absence of the viral genome or surface antigen associated with the viral disease in the virus-infected cell line after the addition of the therapeutic candidate suggests that the therapeutic candidate is an effective agent for the viral disease.

Any of the features described above and herein with regard to virus-infected cell line production methods are applicable to the virus-infected cell lines used herein for screening a therapeutic candidate for a viral disease, which include, but are not limited to, the enveloped virus including a lipid bilayer, the bile acid or bile acid derivative, the immortalized cell line.

As used herein, “viral disease” refers to a disease whose symptoms are caused by viral infection, in which the virus infects a tissue, to which it has tropism, so that a pathological change is induced in the tissue. The viral disease may include any disease as long as the disease causes a pathological change in a tissue. By way of non-limiting example, the viral disease may be viral hepatitis.

As used herein, “viral hepatitis” is hepatitis caused by viral infection, and there are hepatitis B, which accounts for 86% of patients with hepatitis, and hepatitis C, which accounts for 12% of the patients with hepatitis. The cause of the hepatitis B is either vertical transmission in which infection from mother to child occurs at birth, or acquired infection that occurs after birth. For the acquired infection, 95% of infected people have symptoms similar to cold and are naturally cured; however, 5% to 10% of the infected people are not cured and may develop into chronic hepatitis. Conversely, for the vertical transmission, most infected people develop into chronic hepatitis. On the other hand, the hepatitis C is self-limited in only about 10% to 15% of patients, which is relatively small as compared with hepatitis B, and 85% to 90% or higher of the patients develop into chronic hepatitis. In 10% to 20% of the patients with chronic hepatitis, a disease such as cirrhosis or liver cancer develops within 20 to 30 years.

For a viral disease, the infection is identified by checking relevant antigens and antibodies through serological testing. Therefore, to identify whether a therapeutic candidate for a viral disease has a therapeutic effect, the screening method disclosed above and herein detects presence or absence of a viral genome or surface antigen that is associated with the viral disease in the virus-infected cell line after the addition of the therapeutic candidate. In a case where a level of the viral genome or surface antigen present is reduced in the virus-infected cell line model, to which the therapeutic candidate has been added, as compared with the virus-infected cell line without the addition of the therapeutic candidate, the candidate can be selected as a therapeutic agent for the viral disease.

In one embodiment, the identification of presence or absence of a viral genome or surface antigen may be made at a protein or nucleic acid level using methods known in the art. By way of non-limiting example, the detection may be performed through an antigen-antibody reaction, a substrate that specifically binds to a surface antigen, or a reaction with a nucleic acid, a peptide aptamer, or the like.

For hepatitis B, the associated antigens include, but are not limited to, HBsAg antigen (a surface antigen), HBeAg antigen (a secreted antigen), and HBcAg antigen (a core antigen).

By way of non-limiting example, presence or absence of these hepatitis B antigens may be identified by known detection methods such as fluorescence-activated cell sorting, ELISA, Western blotting, time-resolved fluorescence immunoassay (TRFIA), and immunohistochemistry. By way of non-limiting example, presence or absence of the HBV DNA may be examined through gene amplification experiments such as polymerase chain reaction and real-time polymerase chain reaction.

For hepatitis C, by way of non-limiting example, detection of HCV core protein and presence or absence of anti-HCV antibody may be identified through known detection methods, and presence or absence of HCV RNA may be examined through gene amplification experiments such as polymerase chain reaction and real-time polymerase chain reaction.

Still yet another aspect of the present disclosure provides a method for screening a therapeutic candidate for a viral disease by administering a therapeutic candidate for a viral disease to the virus-infected animal model described above and herein. In this method, a decreased or reduced presence or absence of the viral genome or surface antigen associated with the viral disease in the virus-infected animal model post administration of the therapeutic candidate suggests that the therapeutic candidate is an effective agent for the viral disease.

Any of the features described above and herein with regard to virus-infected animal model production methods are applicable to the virus-infected animal model used herein for screening a therapeutic candidate for a viral disease, which include, but are not limited to, the enveloped virus including a lipid bilayer and the bile acid or bile acid derivative.

In one embodiment, a target animal may be a vertebrate animal other than humans, and may be selected from the group consisting of a mouse, rat, rabbit, bird, cat, dog, pig, sheep, goat, deer, horse, and cattle. By way of non-limiting example, the target animal is a mouse or a rat.

In a case where a virus-infected animal model is produced using a vertebrate animal other than humans, the animal model may be used to understand various phenomena such as infection path of the virus and disease development caused by the virus, and deduce significant results therefrom because the animal model is similar to humans in terms of the immune system and the like.

Any disease whose symptoms are caused by viral infection, in which the virus infects a tissue, to which it has tropism, so that a pathological change is induced in the tissue may be considered as a viral disease. The viral disease may include any disease as long as the disease causes a pathological change in a tissue. By way of non-limiting example, the viral disease may be viral hepatitis.

Viral hepatitis is hepatitis caused by viral infection, and there are hepatitis B, which accounts for 86% of patients with hepatitis, and hepatitis C, which accounts for 12% of the patients with hepatitis. The cause of the hepatitis B is either vertical transmission in which infection from mother to child occurs at birth, or acquired infection that occurs after birth. For the acquired infection, 95% of infected people have symptoms similar to cold and are naturally cured; however, 5% to 10% of the infected people are not cured and may develop into chronic hepatitis. Conversely, for the vertical transmission, most infected people develop into chronic hepatitis. On the other hand, the hepatitis C is self-limited in only about 10% to 15% of patients, which is relatively small as compared with hepatitis B, and 85% to 90% or higher of the patients develop into chronic hepatitis. In 10% to 20% of the patients with chronic hepatitis, a disease such as cirrhosis or liver cancer develops within 20 to 30 years.

For a viral disease, the infection is identified by checking relevant antigens and antibodies through serological testing. Therefore, to identify whether a therapeutic candidate for a viral disease has a therapeutic effect, the screening method disclosed above and herein detects presence or absence of a viral genome or surface antigen that is associated with the viral disease in the virus-infected cell line after the addition of the therapeutic candidate. In a case where a level of the viral genome or surface antigen present is reduced in the virus-infected cell line model, to which the therapeutic candidate has been added, as compared with the virus-infected cell line without the addition of the therapeutic candidate, the candidate can be selected as a therapeutic agent for the viral disease.

In one embodiment, the identification of presence or absence of a viral genome or surface antigen may be made at a protein or nucleic acid level using methods known in the art.

By way of non-limiting example, the detection may be performed through an antigen-antibody reaction, a substrate that specifically binds to a surface antigen, or a reaction with a nucleic acid, a peptide aptamer, or the like.

For hepatitis B, the associated antigens include, but are not limited to, HBsAg antigen (a surface antigen), HBeAg antigen (a secreted antigen), and HBcAg antigen (a core antigen). By way of non-limiting example, presence or absence of these hepatitis B antigens may be identified by known detection methods such as fluorescence-activated cell sorting, ELISA, Western blotting, time-resolved fluorescence immunoassay (TRFIA), and immunohistochemistry. By way of non-limiting example, presence or absence of the HBV DNA may be examined through gene amplification experiments such as polymerase chain reaction and real-time polymerase chain reaction.

For hepatitis C, by way of non-limiting example, detection of HCV core protein and presence or absence of anti-HCV antibody may be identified through known detection methods, and presence or absence of HCV RNA may be examined through gene amplification experiments such as polymerase chain reaction and real-time polymerase chain reaction.

Hereinafter, the present disclosure will be described in more detail by way of examples. These examples are provided by way of illustration and not by way of limitation.

### Example 1: Isolation of Hepatitis Virus

Normal serum (hepatitis B virus (HBV)- and hepatitis C virus (HCV)-negative) and hepatitis virus-infected serum (HBV- or HCV-positive) were received from Wonju Severance Christian Hospital (Ethics Committee Approval No. CR316312).

Each of the above-mentioned sera was added to a tube having 3 ml of a cushion buffer that contains 20 g/L of sucrose, 50 mmol/L of Tris-HCl (pH 7.5), and 30 mmol/L of NaCl. The mixture was centrifuged for 1 hour at 220,000 g. Then, the supernatant was removed from the tube, and 5 ml of phosphate-buffered saline (PBS) solution was added to the tube for dilution of HBV or HCV. Then, the HCV RNA or the HBV DNA contained in the dilution was measured through real-time PCR using the Roche's COBAS TaqMan system to determine a concentration of the isolated virus.

### Example 2: Cell Line Culture

The cell lines used in this study were purchased from the Korea Cell Line Bank, and cell culture was performed using the culture medium and culture method specified by the Korea Cell Line Bank.

For rodent cell lines, the following cell lines were used: mouse C57L (The Jackson Laboratory, USA)-derived Hepa1c1c7 cell line or H4-II-E cell line, which is a liver cancer cell line; RAG which is a kidney cancer cell line; EL4 which is an immune T cell line; LC540 which is a testicular cancer cell line; and LA-4 which is a lung cancer cell line.

For human cell lines, the following cell lines were used: HepG2 or Huh-7 which is a liver cancer cell line; A-498 which is a kidney cancer cell line; MDA-MB-231 which is a breast cancer cell line; A549 which is a lung cancer cell line; Jurkat which is an immune T cell line; and HeLa which is a uterine cancer cell line. As a control, PLC/PRF5 cell line, which is a liver cell line, was used.

After thawing, the cells were sub-cultured three times for cell stabilization, and then cultured for 24 hours. Thereafter, hepatitis viral infection was performed.

### Example 3: Production of Virus-Infected Cell Line

For production of enveloped virus-infected cell line models, HBV (1×10⁶ copies/ml/well of HBV) isolated in Example 1 above was mixed with 100 μmol/L of taurochenodeoxycholic acid (hereinafter referred to as ‘tCDCA’), and HCV (1×10⁶ IU/ml/well of HCV) isolated in Example 1 above was mixed with tCDCA or taurocholic acid (hereinafter referred to as ‘tCA’). Here, tCA was mixed with HCV and used to infect mouse hepatocytes; and tCDCA was used in all other cases.

A cell line (HepG2, Huh-7, A549, A-498, MDA-MB-231, Jurkat, HeLa, Hepa1c1c7, EL4, H4-II-E, LA-4, LC540, or RAG cell line) at 5×10⁶ cells/ml was dispensed into each well of a 6-well plate, and culture was performed for 24 hours. Then, the tCDCA or tCA solution mixed with HBV or HCV was added thereto, and culture was further performed for 24 hours. Here, as a control, a group, to which only HBV or HCV was added without the tCDCA or tCA solution, was used. Then, washing with phosphate buffered saline (PBS) solution was performed three times, and the medium was replaced with cell culture medium (DMEM) that does not contain the virus. Finally, the cell culture medium was treated with 100 μmol/L of tCDCA or tCA, and culture was further performed for 3 days, thereby producing a virus-infected cell line model (hereinafter referred to as “infected cell line”).

### Example 4: Verification of Infected Cell Line by HBV

Levels of HBsAg and HBV DNA present were measured in each of the HBV-infected cell lines produced and the culture medium collected therefrom. Here, the measurement of HBsAg was performed using an Elecsys HBsAg II CLIA kit (REF: 07251076119, Roche, Switzerland) by an experimental method provided by the manufacturer, and the measurement of HBV DNA was performed by requesting a specialized inspection agency (Biogeno Korea Co., Ltd., Korea) that utilizes the Roche's COBAS TaqMan system.

In addition, FACS analysis was performed using an anti-HBcAg antibody, to measure a level of HBcAg present. Specifically, 100 μl/well of anti-HBcAg antibody (10E11) (cat no. MA1-7608; Thermo Fisher, USA) was added to a micro ELISA plate, and the reaction was allowed to proceed at 4° C. overnight. Then, 200 μl/well of 1× ELISA/ELISPOT Diluent (Invitrogen, USA) was additionally added thereto, and the reaction was allowed to proceed for 1 hour. Each of the HBV-infected cell lines produced was dispensed into each well, in which the reaction had been completed, and the reaction was allowed to proceed for 2 hours. Then, the anti-HBcAg antibody (10E11) diluted in a ratio of 1:30 with 100 μl of 1× ELISA/ELISPOT Diluent was added to the plate, and the reaction was allowed to proceed for 1 hour. Then, the additional reaction with 100 μl of HRP-conjugated anti-rabbit IgG was allowed to proceed for 30 minutes. For detection of HBcAg, FACSCalibu was used, and the results are shown in Tables 1 and 2. Here, as a positive control, PLC/PRF5 cell line (human liver-derived cell line that releases HBV surface antigen) was used, and levels of HBsAg and HBcAg present therein were measured.

TABLE 1

| | | | | | | MDA- | | |
|---|---|---|---|---|---|---|---|---|
| | Negative | | | | | MB- | | |
| | Control* | PLC/PRF5** | HepG2 | Huh-7 | A498 | 231 | Jurkat | HeLa |
| HBcAg | Not | 32.7% | 61.6% | 69.8% | 67.1% | 87.4% | 37.5% | 89.6% |
| (Index) | detected | | | | | | | |
| HBsAg | 1.00 or | 71.49 ± 1.53 | 474.70 ± 176.30 | 26.02 ± 5.97 | 5.12 ± 0.19 | 169 | 1081 ± 329 | 61.35 |
| (Index) | lower | | | | | | | |
| HBV DNA | 58.2 or | 65,100 ± 9,098 | 3,590,000 ± 1,010,000 | 89,000 ± 1,836 | 73,500 ± 23,292 | 116 | 868,000 ± 88,963 | 359 |
| (copies/ml) | lower | | | | | | | |

*Negative control: HepG2 cell line group, treated with only HBV
**Positive control: Group, not treated with virus and tCDCA

TABLE 2

| | Control*** | Hepa1c1c7 | H4-II-E | RAG | EL4 | LC540 |
|---|---|---|---|---|---|---|
| HBcAg | Not | 77.1% | 32.6% | 76.6% | 66.9% | — |
| (Index) | detected | | | | | |
| HBsAg | 1.00 or | 508.26 ± 107.6 | 22.73 ± 4.39 | 21 (744) | 192.7 ± 30.60 | 3.06 ± 0.44 |
| (Index) | lower | | | | | |
| HBV DNA | 58.2 or | 7,036,667 | 73,000 ± 51,929 | 11,700 (622, 000) | 673,000 ± 60,053 | 149,000 ± 29,866 |
| (copies/ml) | lower | | | | | |

***Control: Hepa1c1c7 cell line group, treated with only HBV

As shown in Tables 1 and 2, it was identified that HBsAg and HBcAg were detected in the culture medium of HepG2, which is a human liver cancer cell line, and Hepa1c1c7, which is a mouse liver cancer cell line, and at least 100 copies of HBV DNA were present therein. Likewise, as a result of checking HBcAg of HBV through FACS, it was identified that the cell lines were infected with the tCDCA-treated HBV as shown at the genome level.

In addition, it was identified that HBsAg and HBcAg were detected in the culture medium of each of cell lines (A498, MDM-MB-231, Jurkat, HeLa, H4-II-E, RAG, EL4, and LC540) derived from various organs other than human and mouse liver, and HBV DNA was present therein.

From the above results, it can be seen that in a case where a cell culture medium of each of human and mouse liver cancer cell lines was treated with a bile acid or a bile acid derivative and HBV together, HBV can be induced to infect the cell lines, indicating that the bile acid or the bile acid derivative can eliminate HBV's species specificity (or narrow host range) and organ tropism so that HBV is induced to infect cell lines derived from various species and tissues other than the liver.

### Example 5: Verification of Infected Cell Line by HCV

The levels of HCV core and HCV RNA present were measured in each of the HCV-infected cell lines produced and the culture medium collected therefrom. Specifically, measurement of HCV RNA was performed by requesting a specialized inspection agency (Biogeno Korea Co., Ltd., Korea) that utilizes the Roche's COBAS TaqMan system. The results are shown in Tables 3 and 4. Here, as a control, a group treated with only HCV without a bile acid derivative was used.

TABLE 3

| | | | | | | MDA- | |
|---|---|---|---|---|---|---|---|
| | | | | | | MB- | |
| | Control | HepG2 | Huh-7 | A549 | A498 | 231 | Jurkat |
| HCV RNA | 15 or | 85.233 | 628.066 | 4550 | 610 | 605 | 3326.667 |
| (IU/ml) | lower | | | | | | |

TABLE 4

| | Control | Hepa1c1c7 | H4-II-E | EL4 | LC540 |
|---|---|---|---|---|---|
| HCV RNA | 15 or lower | 3430 | 197.33 | 1669 | 21.7 |
| (IU/ml) | | | | | |

As shown in Tables 3 and 4, it was identified that the HCV RNA was present at 1000 IU/ml or higher in the culture medium of each of HepG2 cell line, which is a human liver cancer cell line, and Hepa1c1c7 cell line, which is a mouse liver cancer cell line. In addition, it was identified that the HCV RNA was also detected in human- and mouse-derived cell lines that are derived from organs other than the liver.

From the above results, it can be seen that, similar to the results for HBV, in a case where a cell culture medium of each of human and mouse liver cancer cell lines was treated with a bile acid or a bile acid derivative and HCV together, HCV can be induced to infect the cell lines. Furthermore, it can be seen that the bile acid or the bile acid derivative can eliminate HCV's species specificity or narrow host range and organ tropism so that HCV is induced to infect cell lines derived from various species and tissues.

The above results also show that in a case where a cell line is treated with a bile acid or a bile acid derivative and HBV or HCV together, the bile acid or the bile acid derivative expands the lipid bilayer of the cell line, thereby causing mechanical destruction of the cell membrane, so that the hepatitis viral infection can be very effectively induced.

### Example 6: Production of Virus Derived from Mouse Liver Cancer Cell Line

For production of an enveloped virus-infected animal model, HBV (1×10⁸ copies/ml/well of HBV) or HCV (1×10⁸ IU/ml/well of HCV) isolated in Example 1 above was mixed with 100 μmol/L of tCDCA.

Hepa1c1c7 cell line at 1×10⁵ cells/ml was dispensed into a 6-well plate, and culture was performed for 24 hours. Then, the tCDCA solution mixed with HBV or HCV was added thereto, and culture was further performed for 24 hours. Here, as a control, a group to which only the tCDCA solution was added was used. Then, washing with PBS solution was performed three times, and the medium was replaced with a cell culture medium that does not contain the virus. Subsequently, culture was further performed for 3 days. Then, viruses were isolated from the culture medium obtained from the Hepa1c1c7 cell line in the same manner as described in Example 1 above.

Here, in the isolated viruses, the lipid bilayer envelope was replaced with one derived from the Hepa1c1c7 cell line. Thus, the viruses were designated mouse liver cancer cell line-derived HBV (hereinafter referred to as ‘cHBVcc’) and mouse liver cancer cell line-derived HCV (hereinafter referred to as ‘cHCVcc’), respectively. Then, the concentration of the respective viruses was checked through real-time PCR using the COBAS TaqMan system (Roche, Switzerland).

### Example 7: Viral Stability of cHBVcc and cHCVcc

Viral stability of cHBVcc and cHCVcc was checked in various environments, before identifying their cross-infectivity. Human serum (HS), mouse serum (MS), and a culture medium of a cell line were treated with the cHBVcc or cHCVcc, and then levels of HBsAg and HBV DNA present in the sera or the culture medium were measured as described above. The results are shown in Table 5. Here, as a control, the serum of a patient with chronic hepatitis was used.

TABLE 5

| | Control | Human serum | Mouse serum | Cell culture medium |
|---|---|---|---|---|
| HBsAg | 2,897 | Negative(negative) | Negative | Negative |
| (Index) | | | | |
| HBV DNA | 4.36 × 10⁸ | 224,000 | 31,400 | 12,300 |
| (copies/ml) | | | | |

As shown in Table 5, it can be seen that as compared with the control, the cHBVcc, for which replacement with a mouse liver cancer cell line-derived lipid bilayer had been achieved, exhibited negative surface antigenicity and also had a greatly decreased amount of genome; however, the amount of genome corresponds to a value well above the minimum infectious dose, and thus even the cHBVcc shows stability in terms of viral envelope state, so that the cHBVcc can very effectively infect a target animal such as humans or mice.

### Example 8: Identification of Viral Cross-Infection

Identification of cross-infectivity of cHBVcc: Cross-infectivity of the cHBVcc obtained above was identified in Hepa1c1c7 cell line. Specifically, the Hepa1c1c7 cell line was treated with a mixed solution (cHBVcc+tCDCA), obtained by mixing the cHBVcc with 100 μmol/L of tCDCA, or cHBVcc, and then HBsAg and HBV DNA were measured as described above. The results are shown in Table 6. Here, as a control, the HBV (wild-type HBV) obtained in Example 1 above was used.

TABLE 6

| | Wild-type HBV | HBV + tCDCA | cHBVcc | cHBVcc + tCDCA |
|---|---|---|---|---|
| HBsAg | Negative | 18.61 | 7.61 | Negative |
| (Index) | | | | |
| HBV DNA | Negative | 657 | 190,000 | 130,000 |
| (copies/ml) | | | | |

As shown in Table 6, it was identified that in the Hepa1c1c7 cell line, no infection occurred in a case of being treated with the wild-type HBV, and infection occurred in a case of being treated with a mixture with tCDCA.

Furthermore, it was identified that in the Hepa1c1c7 cell line, infection with cHBVcc occurred in a case of being treated with the cHBVcc whose lipid bilayer had been replaced with one derived from the Hepa1c1c7 cell line even in the absence of tCDCA (cHBVcc).

From the above results, it can be seen that even in an environment where a bile acid or a bile acid derivative is not present, the virus, for which replacement of lipid bilayer envelope has been achieved using a bile acid or a bile acid derivative, can infect other allogeneic cells, and furthermore, can very effectively infect an animal corresponding to the same species as the cells.

Identification of cross-infectivity of cHCVcc: Cross-infectivity of the cHCVcc obtained above was identified in Hepa1c1c7 cell line. Specifically, the Hepa1c1c7 cell line was treated with a mixed solution (cHCVcc+tCDCA), obtained by mixing the cHCVcc with 100 μmol/L of tCDCA, or cHCVcc, and then HCV RNA was measured as described above. The results are shown in Table 7. Here, as a control, the HCV (wild type HCV) obtained in Example 1 above was used.

TABLE 7

| | Wild-type HCV | HCV + tCDCA | cHCVcc | cHCVcc + tCDCA |
|---|---|---|---|---|
| HCV RNA | Negative | 3,403 | 1330 | 1330 |
| (IU/ml) | | | | |

As shown in Table 7, it was identified that in the Hepa1c1c7 cell line, no infection with the wild-type HCV occurred in a case of being injected with only the wild-type HCV, and infection occurred in a case of being treated with a mixture with tCDCA.

Furthermore, it was identified that in the Hepa1c1c7 cell line, infection occurred in a case of being treated with the cHCVcc whose lipid bilayer had been replaced with one derived from the Hepa1c1c7 cell line even in the absence of tCDCA.

From the above results, it can be seen that even in an environment where a bile acid or a bile acid derivative is not present, the virus, for which replacement of lipid bilayer envelope has been achieved using a bile acid or a bile acid derivative, can infect xenogeneic cells, and furthermore, can very effectively infect an animal whose species is the same as that from which the cells are derived.

To further identify cross-infectivity, a solution obtained by mixing HBV (1×10⁸ copies/ml/well of HBV) or HCV (1×10⁸ IU/ml/well of HCV) isolated in Example 1 above with 100 μmol/L of tCDCA was added to HepG2 cell line. HBV and HCV for which replacement of lipid bilayer envelope had been achieved were then obtained according to the method described above. HepG2 cell line was further treated with 100 μmol/L of bile acid-mixed solution (HBV+tCDCA or HCV+tCDCA), and levels of HBsAg and HBV DNA or HCV RNA were then measured in the same manner as described above. The results are shown in Tables 8 and 9.

TABLE 8

| | HBV | HBV + tCDCA |
|---|---|---|
| HBsAg | 5.99 ± 0.89 | 4.67 ± 0.58 |
| (Index) | | |
| HBV DNA | 48000 ± 18504 | 76400 ± 19552 |
| (copies/ml) | | |

TABLE 9

| | HCV | HCV + tCDCA |
|---|---|---|
| HCV RNA | 15 or lower | 182 |
| (IU/ml) | | |

As shown in Table 8, in the presence of tCDCA, the HepG2 cell line was infected with the HBV whose lipid bilayer envelope is derived from the HepG2 cell line, and thus HBsAg and HBV DNA were detected therein. Furthermore, as shown in Table 9, it was identified that the HepG2 cell line was infected with the HCV whose lipid bilayer envelope is derived from the HepG2 cell line, and thus 182 IU/ml of RNA was present therein.

From the above results, it can be seen that the virus, for which replacement of lipid bilayer envelope has been achieved using a bile acid or a bile acid derivative, can very efficiently infect allogeneic cells.

Next, a solution obtained by mixing HBV (1×10⁸ copies/ml/well of HBV) or HCV (1×10⁸ IU/ml/well of HCV) isolated in Example 1 above with 100 μmol/L of tCDCA was added to HepG2 cell line. HBV and HCV for which replacement of lipid bilayer envelope had been achieved were then obtained according to the method described above. Hepa1c1c7 cell line was treated with 100 μmol/L of bile acid-mixed solution (HBV+tCDCA or HCV+tCDCA), and levels of HBsAg and HBV DNA or HCV RNA were then measured in the same manner as described above. The results are shown in Tables 10 and 11.

TABLE 10

| | HBV | HBV + tCDCA |
|---|---|---|
| HBsAg | 3.28 ± 0.89 | 19.32 ± 1.85 |
| (Index) | | |
| HBV DNA | 47000 ± 13200 | 292000 ± 38553 |
| (copies/ml) | | |

TABLE 11

| | HCV | HCV + tCDCA |
|---|---|---|
| HCV RNA | 15 or lower | 182.3 |
| (IU/ml) | | |

As shown in Tables 10 and 11, in the presence of tCDCA, the Hepa1c1c7 cell line was infected with the HBV whose lipid bilayer envelope is derived from the HepG2 cell line, and thus HBsAg and HBV DNA were detected therein. Furthermore, as shown in Table 11, it was identified that the Hepa1c1c7 cell line was infected with the HCV whose lipid bilayer envelope is derived from the HepG2 cell line, and thus 182.3 IU/ml of RNA was present therein.

From the above results, it can be seen that the virus, for which replacement of lipid bilayer envelope has been achieved using a bile acid or a bile acid derivative, can infect xenogeneic cells, and further can infect an animal whose species is different from that from which the cells are derived.

Still next, a solution obtained by mixing HBV (1×10⁸ copies/ml/well of HBV) or HCV (1×10⁸ IU/ml/well of HCV) isolated in Example 1 above with 100 μmol/L of tCDCA was added to Hepa1c1c7 cell line. The same method as described above was performed to obtain HBV and HCV for which replacement of lipid bilayer envelope had been achieved. HepG2 cell line was treated with 100 μmol/L of bile acid-mixed solution (HBV+tCDCA or HCV+tCDCA), and levels of HBsAg and HBV DNA or HCV RNA were then measured in the same manner as described above. The results are shown in Tables 12 and 13.

TABLE 12

| | HBV | HBV + tCDCA |
|---|---|---|
| HBsAg | Not detected | 32.5 ± 1.85 |
| (Index) | | |
| HBV DNA | 15.8 or lower | 392000 ± 32203 |
| (copies/ml) | | |

TABLE 13

| | HCV | HCV + tCDCA |
|---|---|---|
| HCV RNA | 15 or lower | 300 |
| (IU/ml) | | |

As shown in Table 12, in the presence of tCDCA, the HepG2 cell line was infected with the HBV whose lipid bilayer envelope is derived from the Hepa1c1c7 cell line, and thus HBsAg and HBV DNA were detected therein. Furthermore, as shown in Table 13, it was identified that the Hepa1c1c7 cell line was infected with the HCV whose lipid bilayer envelope is derived from the HepG2 cell line, and thus 300 IU/ml of RNA was present therein.

From the above results, it can be seen that the virus, for which replacement of lipid bilayer envelope has been achieved using a bile acid or a bile acid derivative, can very efficiently infect allogeneic cells.

### Example 9: Production and Verification of HBV- and HCV-Infected Animal Models

The cHBVcc or cHCVcc prepared using the methods described above was injected in an amount of 100 μl (1×10⁸ genome equivalents) into the tail vein of mouse C57L, and the mouse was reared for 1 week to produce a virus-infected animal model (hereinafter referred to as “infected animal”). A mouse into which human-derived wild-type HBV was injected in an amount of 100 μl was used as a control.

Virus test in serum: the serum was obtained from each infected animal, and levels of HBsAg and HBV DNA present in the serum were measured. The serum from a mouse into which wild-type HBV having a human-derived lipid bilayer envelope was injected was used as a control. HBsAg and HBV DNA or HCV RNA were then measured according to the manner described above. The results are shown in Tables 14 and 15.

TABLE 14

| | Control | mHBVcc |
|---|---|---|
| HBsAg | 0.1 (negative) | 1.2 |
| HBV DNA | 58.2 or lower | 585 |
| (copies/ml) | (negative) | |

TABLE 15

| | Control | mHCVcc |
|---|---|---|
| HCV RNA | 15 or lower | 180 |
| (IU/ml) | | |

As shown in Table 14, it was identified that the level of HBsAg in the infected animal's serum was 1.2 Index, and HBV DNA was present in 585 copies/ml. In addition, as shown in Table 15, it was identified that for HCV RNA, the control exhibited 15 IU/ml or lower, whereas the mHCVcc exhibited 180 IU/ml.

From the above results, it can be seen that injection of cHBVcc and cHCVcc can induce HBV infection in a small animal, e.g., mouse, which is species other than humans.

Liver function test: to check whether hepatitis caused by viral infection had occurred in the infected animal, a liver function test, in which a level of alanine aminotransaminase (ALT) in serum is measured, was performed by a conventional method known in the art. ALT in the infected animal injected with cHBVcc was measured to be 150 U/L.

From the above results, it can be seen that since the normal ALT value has 34 or lower, injection of cHBVcc can cause HBV infection in the mouse that is an infected animal, indicating that hepatitis has occurred in the infected animal.

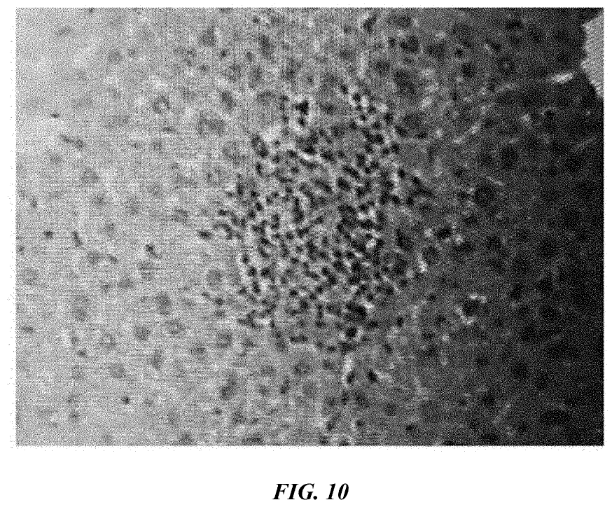

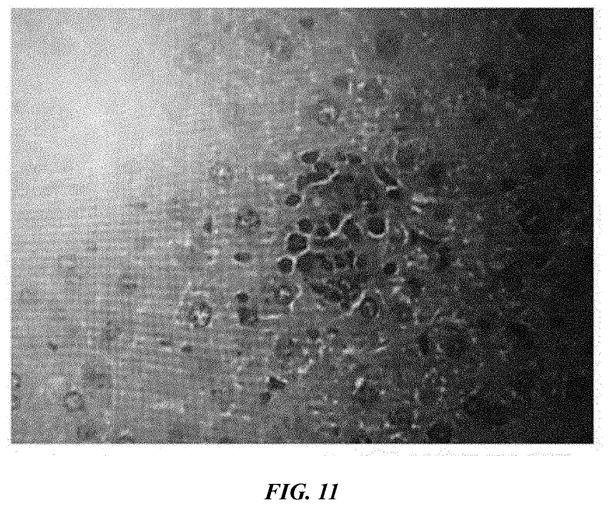

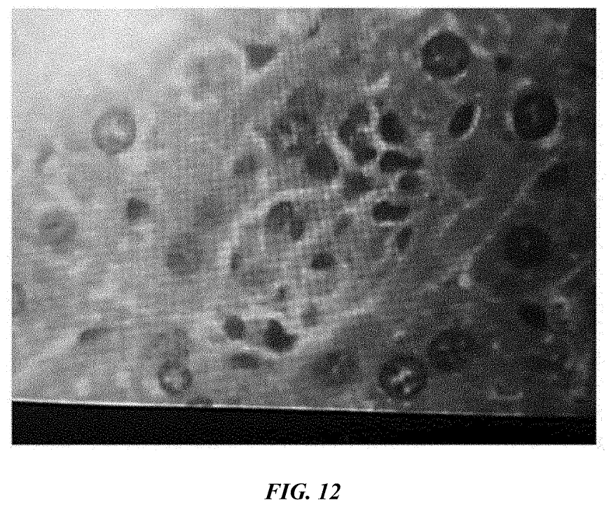

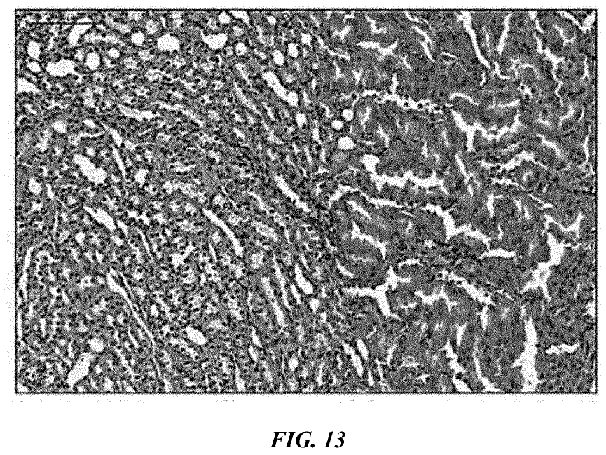

Tissue biopsy: liver tissues were excised from the infected animal, fixed using 10% formalin, and then embedded in paraffin. The paraffin-embedded tissue was cut into 4 μm-thick sections, and rehydration and paraffin removal were performed. Then, H&E staining was performed for the sections through a conventional method, and the results are illustrated in FIGS. 9-16.

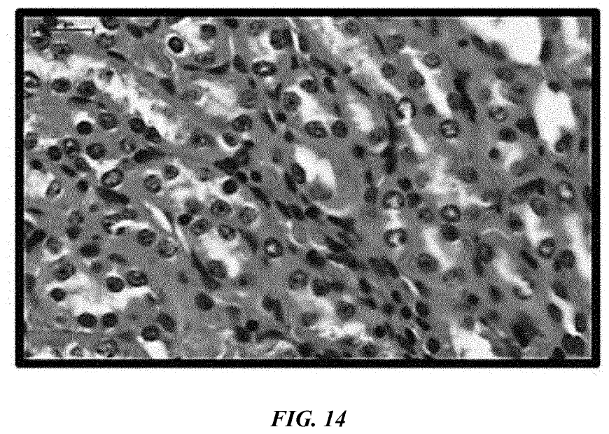

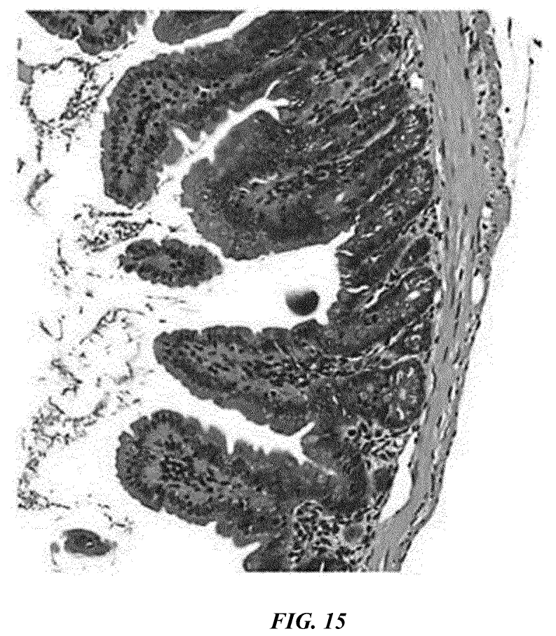

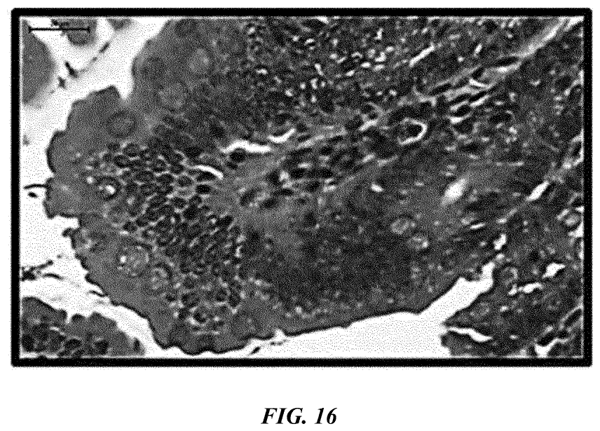

As shown in FIG. 9 and FIG. 14, it was identified that inflammation occurred in view of the fact that increased infiltration of monocytes (lymphocytes) was observed at several places in the hepatic parenchyma. Infiltration of monocytes and balloon-shaped mass were also observed in the peri-portal area, which was similar to histological findings of human acute hepatitis B. As shown in FIGS. 15-16, it was identified that monocytes (lymphocytes) were infiltrated into the villi of the ileum.

From the above results, it can be seen that the cHBVcc can infect a rodent and such an infection can lead to acute inflammation caused by HBV infection in renal and iliac tissues as well as liver tissues.

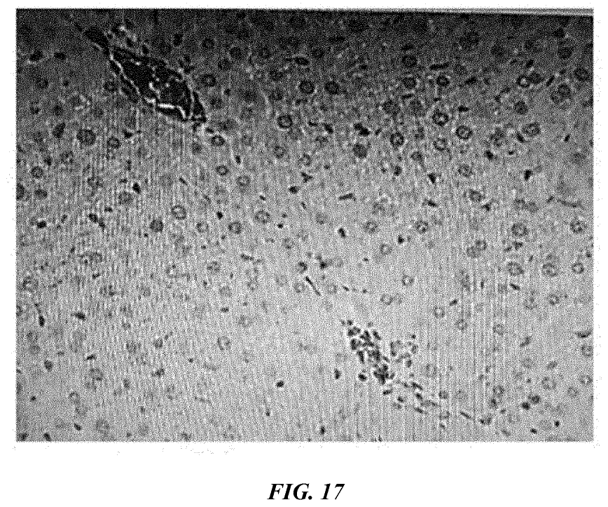

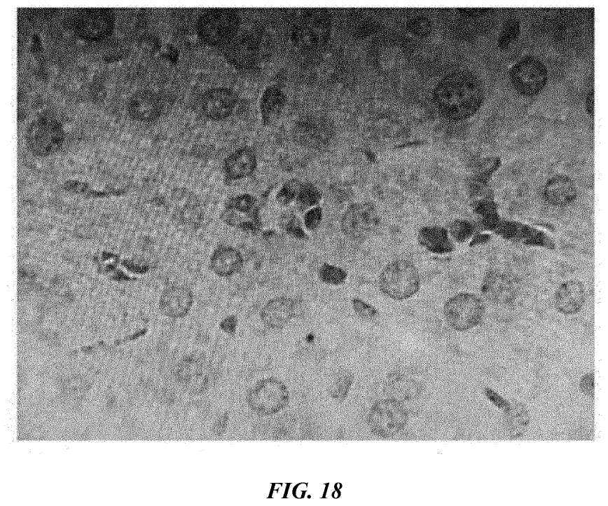

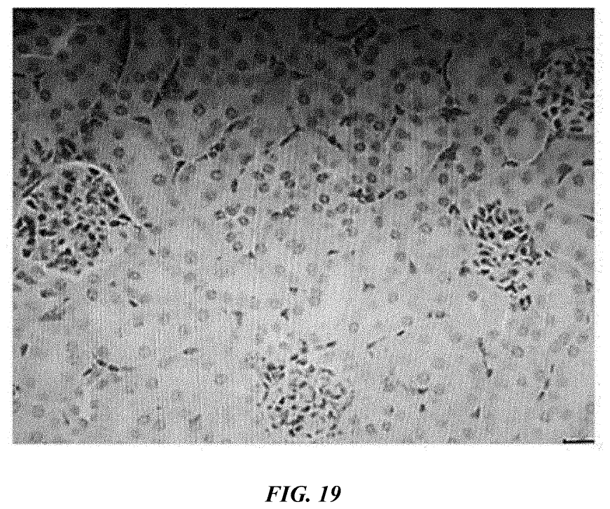

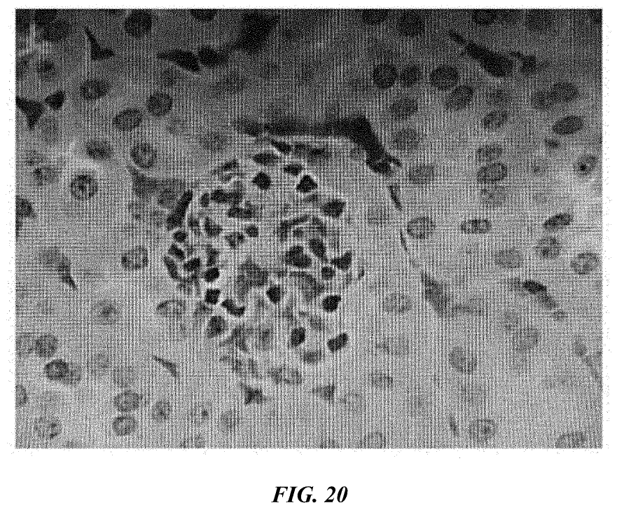

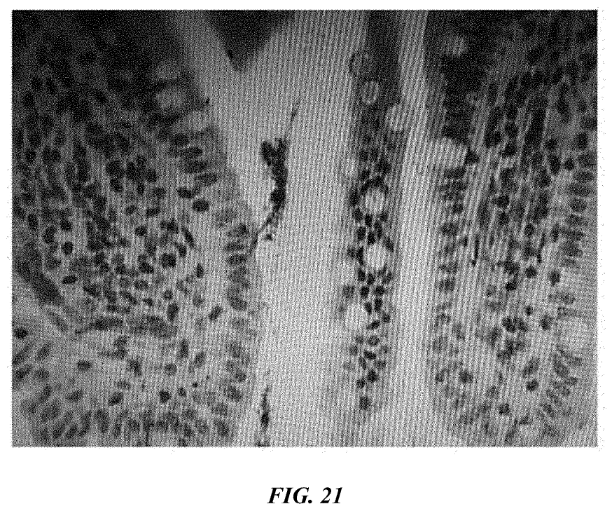

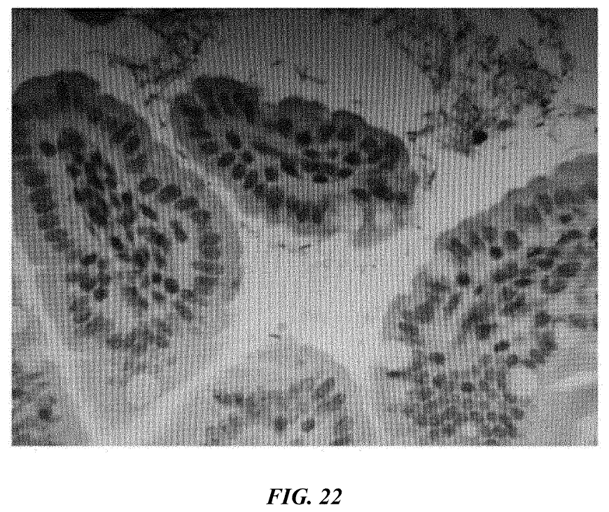

Immunostaining test: liver tissues were excised from the infected animal, fixed using 10% formalin, and then embedded in paraffin. The paraffin-embedded tissue was cut into 4 μm-thick sections, and rehydration and paraffin removal were performed. Then, the tissue was reacted with an antibody against HBcAg, which is an HBV core antigen, and reacted with a secondary antibody to which a substance (biotin) that can be visualized is bound. Then, staining with Hoechst 33258 was performed for 15 minutes. Then, mounting was performed through 50% glycerol, and observation was performed under a microscope. The results are shown in FIGS. 17-22.

As illustrated therein, the cytoplasm was stained brown in hepatocytes, kidney cells, and ileal villous cells. This suggests that by using the method for producing an animal model as described above and herein, it is possible to produce an efficient animal model that can be effectively used clinically without limitation resulting from species specificity, because a human-derived viral lipid bilayer is replaced with a target animal model-derived lipid bilayer.

In conclusion, for a human-derived lipid bilayer of an enveloped virus, a bile acid or a bile acid derivative allows the lipid bilayer to be replaced with a target animal model-derived lipid bilayer, thus makes it possible to produce virus-infected cell line or animal model which can be effectively used clinically without limitation resulting from species specificity. Accordingly, there is an industrial applicability in that the cell lines and the animal models produced above and herein can be used to effectively screen a therapeutic candidate for a disease caused by a virus including a lipid bilayer envelope, thereby remarkably reducing costs associated therewith.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims. No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1-27. (canceled)

28. A method for producing a virus-infected cell line, the method comprising:
(1) mixing an enveloped virus including a lipid bilayer with a bile acid or a bile acid derivative; and
(2) adding the virus from (1) to a culture medium of a cell line, thereby producing a virus-infected cell line.

29. The method of claim 28, wherein the enveloped virus is a hepatitis virus.

30. The method of claim 29, wherein the hepatitis virus is hepatitis B virus (HBV) or hepatitis C virus (HCV).

31. The method of claim 28, wherein the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid, or a combination thereof.

32. The method of claim 28, wherein the cell line is selected from the group consisting of a liver cell line, a lung cell line, a kidney cell line, a muscle cell line, a breast cell line, an immune T cell line, and a reproductive cell line.

33. The method of claim 32, wherein the cell line is derived from human or mouse.

34. The method of claim 33, wherein the cell line is human liver cancer cell line HepG2, mouse liver cancer cell line Hepa1c1c7, human lung cancer cell lines SNU-1327 and A549, mouse lung cancer cell line LA-4, human kidney cancer cell line A498, mouse kidney cancer cell line RAG, human breast cancer cell line MDA-MB-231, mouse breast cancer cell line MTV/TM-011, human immune T cell line Jurkat, mouse immune T cell line EL4, human cervical cancer cell line HeLa, or mouse testicular cancer cell line LC540.

35. A virus-infected cell line, produced by the method of claim 28.

36. A method for producing a virus-infected animal model, the method comprising:
(1) replacing a lipid bilayer of an enveloped virus with a lipid bilayer derived from a target animal; and
(2) injecting the virus for which the lipid bilayer replacement has been achieved into the target animal, thereby producing a virus-infected animal model.

37. The method of claim 36, wherein the enveloped virus is mixed with a bile acid or a bile acid derivative in step (1), thereby replacing a lipid bilayer of the enveloped virus with a lipid bilayer derived from the target animal.

38. The method of claim 36, wherein the target animal is a vertebrate animal selected from the group consisting of a mouse, rat, rabbit, bird, cat, dog, pig, sheep, goat, deer, horse, and cattle.

39. The method of claim 38, wherein the target animal is a mouse.

40. The method of claim 36, wherein the enveloped virus is a hepatitis virus.

41. The method of claim 40, wherein the hepatitis virus is hepatitis B virus (HBV) or hepatitis C virus (HCV).

42. The method of claim 37, wherein the bile acid or bile acid derivative is selected from the group consisting of cholic acid, chenodeoxycholic acid, deoxycholic acid, lithocholic acid, taurocholic acid, glycocholic acid, taurochenodeoxycholic acid, glycochenodeoxycholic acid, glycodeoxycholic acid, taurodeoxycholic acid, glycolithocholic acid, taurolithocholic acid, and muricholic acid, or a combination thereof.

43. A virus-infected animal model, produced by the method of claim 36.
